(19) United States
(12) Patent Application Publication
(10) Pub. No.: US 2026/0076723 A1
(43) Pub. Date: Mar. 19, 2026

(54) Orthopedic Screw

(71) Applicant: 41MEDICAL AG, Bettlach (CH)

(72) Inventors: Philipp MORODER, Meilen (CH); Michelle STUTZ, Bachenbülach (CH); Silas ZURSCHMIEDE, Bettlach (CH); Alfred NIEDERBERGER, Grenchen (CH)

(73) Assignee: 41MEDICAL AG, Bettlach (CH)

(21) Appl. No.: 19/135,317

(22) PCT Filed: Dec. 8, 2023
(86) PCT No.: PCT/EP2023/084932

§ 371 Date: Jun. 3, 2025

(30) Foreign Application Priority Data
Dec. 8, 2022 (CH) CH001463/2022

(51) Int. Cl. A61B 17/80; A61B 17/00; A61B 17/86

(52) U.S. Cl. CPC A61B 17/8061; A61B 17/863; A61B 17/8635; A61B 2017/00004; A61B 2017/00477; A61B 2017/00526

(57) ABSTRACT

An orthopedic screw includes a threaded shaft made of a non-absorbable material and a tip made of an absorbable material. The shaft and the tip are positively connected.

### CROSS-REFERENCE TO RELATED APPLICATIONS

This application is a U.S. National Stage Application that claims priority to and the benefit of the filing date of International PCT Application No. PCT/EP2023/084932, filed on Dec. 8, 2023, that claims priority to Swiss Application No. CH001463/2022, filed on Dec. 8, 2022, each of which are hereby incorporated by reference in their entirety.

### TECHNICAL FIELD

The present disclosure relates to an orthopedic screw and to a method for manufacturing an orthopedic screw.

### BACKGROUND

A proximal humerus fracture is an injury to the humerus bone in the shoulder joint that often requires immediate treatment to preserve function of the shoulder. A fracture to the humerus bone is typically a consequence of a traumatic event, such as a fall or forceful collision. Fractures of the proximal humerus are common, and they account for approximately 5% of all fractures. These fractures tend to occur in older female patients who are osteoporotic. The most common cause for these fractures is a fall on the outstretched hand from a standing height.

A common way of treating proximal humerus fractures involves using a bone plate, in conjunction with bone screws, thus forming a bone plate assembly. Such an assembly typically has a good functional outcome. However, sintering of the bone often results in screw penetration of the articular or joint surface (also known as a secondary screw penetration) after a certain time, resulting injury of the joint surface and a restricted movement of the joint.

In order to address this problem, cannulated bone screws are used in the prior art, which allow polymethyl methacrylate (PMMA) cement be placed at the tip of the bone screws. This kind of arrangement leads to a very stable construct. However, there is a risk of leaking the PMMA cement into the shoulder joint. To avoid this, the surgical technique typically involves test for possible leakage using a contrast agent. This is followed, if necessary, by use of a saline solution to wash away the contrast agent, before application of the PMMA cement. Therefore, this approach leads to a prolonged surgery and to relatively high costs.

It is a problem underlying the present disclosure to address the above-mentioned shortcomings in the prior art. In particular, the present disclosure shall provide means for an improved treatment of proximal humerus fractures (especially with regard to patient safety and cost), where the risk of secondary screw penetration is substantially reduced.

### SUMMARY

The present disclosure relates to an orthopedic screw. This screw comprises a threaded shaft made of a non-absorbable material and a tip made of an absorbable material. The shaft and the tip are positively connected.

In the present context, the expressions “absorbable material” and “bioabsorbable material” are used as synonyms. A bioabsorbable material is a material which can be used in orthopedic implants, in particular orthopedic screws, and is absorbed by the body over time.

A “positive connection”, also known as “from closure”, as opposed to a “forced closure”, is a connection, which is created by the interlocking of at least two connection partners. As a result, the connection partners cannot disengage even without or with interrupted force transmission.

Orthopedic screws according to the present disclosure have the advantage that they provide good stabilization of a proximal humerus fracture in the initial phase after surgery. In particular, during this period, penetration of the screws into the second cortex adjacent to the humeral head is desired to ensure good stability. As healing of the bone progresses, the tips of the screws are absorbed at approximately the same rate as the bone regrows. Once the healing process is finished, the screws have significantly shortened, because their tips have been substantially or entirely absorbed. The risk of implant-related, long-term complications, in particular secondary screw penetration upon sintering of the bone, is therefore eliminated or at least strongly reduced. For this reason, a surgeon can be more daring in placing the tip of the screw further into the second cortex and closer to the humeral head, i.e. using a longer screw. This further increases stability. Since only temporary stabilization of the humerus bone is needed, the solution according to the present disclosure is superior over conventional systems. Furthermore, the proposed orthopedic screws are as simple to use as conventional screws.

A further advantage of the present disclosure resides in the positive connection between the shaft and the tip, which avoids that the tip prematurely comes lose during placement of the screw or during the initial healing phase. This can be particularly important, if a surgeon has to remove a screw from a bone, which has already been placed. In such a situation, significant tensile forces can act on the tip. There is thus a risk that the tip disconnects from shaft and remains in the bone, as the shaft is retracted. Positive connection between the shaft and the tip essentially eliminates this risk.

With regard to the screw presently discussed, the shaft thread defines a first lead length and a first pitch. Lead length or simply lead is the linear travel the screw makes per one screw revolution. The pitch and lead are equal with single start screws. For multiple start screws the lead is the pitch multiplied by the number of starts. In other words, the pitch equals the lead length divided by the number of thread starts.

In an orthopedic screw according to the present disclosure, the shaft lead length can be in a range of 0.2 mm to 5.0 mm, preferably 0.4 mm to 3.0 mm, more preferably 0.8 mm to 2.0 mm.

The shaft and the tip can be additionally connected by a forced closure, in particular a press-fit connection.

In one aspect of the present disclosure, a proximal end of the tip comprises a recess into which a protrusion at a distal end of the shaft protrudes. This allows for a stable connection of the tip to the shaft.

In the present context, a “proximal end” is understood as the end of the screw, shaft or tip, which is closer to the user of the screw, i.e. the surgeon. A “distal end” is understood as the opposite end, in other words the end away from the user of the screw. Thus, the “distal end” of the screw or tip is arranged to first enter the target bone when implanting the screw. Moreover, the words “distal end” and “proximal end” are understood not to cover a specific end point but also more broadly the respective end region.

In another alternative aspect of the present disclosure, a distal end of the shaft comprises a recess into which a protrusion at a proximal end of the tip protrudes. Also in this way the tip can be stably mounted to the shaft. Additionally, this configuration has the advantage that, as absorption of the tip in the body is ongoing, the part of the tip fixing it at the shaft is covered by the shaft, retarding bioabsorption. This way, it can be avoided that the tip prematurely comes lose from the shaft.

In the present context, the word “recess” is not necessarily restricted to a bind hole, although this is one preferred option. Generally, a “recess” can be any hole, channel or cavity, whether passing through or not, as long as it is adapted for fully or partially receiving the protrusion.

The recess can extend along a longitudinal center axis A1 of the shaft.

The recess can be cylindrical, prismatic or conical in shape. Preferably, the recess is cylindrical. A cylindrical or conical recess has the advantage that, if the tip is pre-manufactured and mounted to the shaft, this can be achieved irrespective of the angular orientation of the tip, relative to the longitudinal center axis A1.

When the recess is prismatic in shape, a cross-section of the recess, to which the longitudinal center axis A1 is perpendicular, can be tetragonal, pentagonal, hexagonal or octagonal. This has the advantage that the tip can be mounted in a rotationally locked manner to the shaft. This is of particular relevance if also the tip is threaded (see further below).

The recess, in particular a sidewall of the recess, can comprise at least one, preferably a plurality of, undercuts. These undercuts can engage with at least one, preferably a plurality of, engagement elements at the protrusion of the tip. This is a preferred way for achieving a positive connection between the tip and the shaft.

In a particular embodiment of the present disclosure, the recess is cylindrical and comprises at least one, preferably a plurality of, undercuts in the form of circumferential grooves. The at least one, preferably a plurality of, engagement elements can then be realized as increased diameter regions of a cylindrical protrusion of the tip. A plurality of undercuts has the advantage that good holding of the tip to the shaft is ensured, even during the degradation process. The number of circumferential grooves can be 1, 2, 3, 4, 5, 6, 7, 8, 9 or 10. A number of 2, 3 or 4 grooves is preferred, in order to achieve a good equilibrium between stability and cost of manufacture. In a preferred embodiment, the at least one undercut in the form of a circumferential groove is formed by turning.

When the shaft comprises a recess, this recess of the shaft can comprise at least one, preferably a plurality of, ventilation holes and/or injection points. Ventilation holes and/or injection points are of particular relevance, if the tip is not provided separately and mounted on the shaft, but injected upon the shaft by injection molding, which is described in more detail herein below.

The at least one ventilation hole and/or injection point can be oriented in a transversal and/or longitudinal, preferably transversal, direction, relative to the shaft. Transversal ventilation holes and/or injection points have the advantage that they can at the same time fulfill the function of an undercut for securing the tip on the shaft.

Furthermore, the at least one ventilation hole and/or injection point can be located in the shaft thread's grooves. This has the advantage that the thread is not interrupted. Furthermore, if the ventilation hole and/or injection point is formed on the threads by drilling or milling, placing the hole in the shaft thread's grooves allows for better control of this process.

There can be an area around the ventilation hole and/or injection point which is devoid of screw threads. In embodiments, where the tip is injected upon the shaft by injection molding, such an area can form a sealing surface to form a thigh connection with an injection mold. The sealing surface can extend cross-shaped, circularly or oval around the hole, or it can be formed by the base of a u-shaped longitudinal groove extending substantially parallel to the center axis A1 of the screw. The sealing surface can have a shortest extension, which is at least three times the diameter of the ventilation hole and/or injection point, preferably four times, even more preferably five times.

The shaft can further comprise a sealing surface in form of a circumferential area at a distal end of the shaft, in order to create a tight connection between the shaft of and an injection mold. The sealing surface can have a cylindrical or conical shape, preferably cylindrical. If the sealing surface is cylindrical, it can have a diameter, which is slightly smaller than the inner diameter of the shaft thread D2. This avoids unnecessary friction, when the screw is screwed into a patient's bone. By way of example, the diameter of the cylindrical sealing surface can be smaller by 0.1 mm.

Furthermore, the cylindrical sealing surface can have a width in a direction parallel to the center axis A1 of 0.4 mm to 4 mm, preferably 1 mm to 2 mm, for instance 1 mm. On one hand, the width of the sealing surface should be sufficient to ensure proper sealing. On the other hand, the sealing surface should not be too wide, in order not to compromise the length of the shaft's threading. Furthermore, extensive width of the sealing surface can lead to an undesired excessive capillary effect.

In cases where the tip is provided separately from shaft and mounted on the same, the shaft and the tip can be connected through a snap fitting. In such cases, a snap fitting is a preferred connection method.

Alternatively, the shaft and the tip can be connected through a thread. A threaded connection is a further option for mounting a pre-manufactured tip on the shaft. An advantage of this method is that the tip can be removed from the shaft with relative ease. Furthermore, if the outer shaft thread has the same sense of rotation as the outer protrusion tip thread, the tip cannot come of the shaft, when the screw is placed into a bone. However, there a certain risk that the tip remains in a bone, if the screw is later removed from the bone.

As a further alternative, the shaft and the tip can be connected to each other by ultrasonic welding. In such a process, a defined area of the tip is molten by ultrasound after insertion in to the shaft's recess, in order to fill an undercut in the side wall of the recess.

The distal end of the shaft can be rounded or chamfered. This means that even after the tip has been fully absorbed, the shaft of the screw still has dull edges, at least at its distal end. With these dull edges, any risk of secondary screw penetration is still reduced. Furthermore, a rounded or chamfered distal end of the shaft has the advantage that a transitional surface between tip and the shaft can be curved or edged and therefore larger. It has been found that this leads to a slowed bioabsorption at the connection between the tip and the shaft, which can help to avoid that the tip prematurely comes lose from the shaft.

In context of the present disclosure, any “rounded”, or in other words curved, surfaces do not have to be spherically rounded. More specifically, any rounded or curved surface may form a section or portion of an ellipsoid or a sphere, for instance.

The tip can be non-threaded. Since for fixation of a proximal humerus fracture pilot holes are normally drilled into a patient's bone, the orthopedic screw according to the present disclosure does not need to be self-tapping. Consequently, the tip can be non-threaded. A further advantage of a non-threaded tip is that there are no sharp edges, which can potentially lead to secondary screw penetration.

The cross-sectional diameter of the pilot hole normally equals or substantially equals the inner diameter of the shaft thread D2, or the diameter of the pilot hole may be slightly less.

The diameter D1 of the tip can be equal to or smaller than the inner diameter D2 of the shaft thread. This has the advantage that the screw can be screwed into a pre-drilled pilot hole without undue burden. Furthermore, there is no or only a small radial force exerted on the tip, then the screw is screwed into the pilot hole. This avoids that the stability of the tip is compromised, either mechanically or through friction heat.

Alternatively, the diameter D1 of the tip can be equal to or smaller than the outer diameter D3 of the shaft thread.

The distal end of the tip can be rounded, i.e. it can be devoid of any edges. This has the advantage that the risk of secondary screw penetration can be further reduced.

The radius of curvature at any point of the distal end of the tip may be >⅕ of the diameter D1 of the tip, preferably >¼, more preferably >⅓, even more preferably >½.

In a specific embodiment, the distal end of the tip can be flat or substantially flat, and a circumferentially rounded non-cylindrical surface extends from the flat tip towards the proximal end of the tip to merge with a cylindrical section.

In an alternative embodiment of the present disclosure, the tip can be threaded. The tip thread then defines a second lead and a second pitch. A threaded tip has the advantage that the holding of the screw in its distal region is improved.

The lead of the shaft thread and the lead of the tip thread can be substantially the same, preferably exactly the same. This has the advantage that the screw can be smoothly screwed into a bone, with any damage caused to the bone tissue being minimized or avoided.

In a particular version of this embodiment, the outer diameter D3 of the shaft thread is the same as the outer diameter of the tip thread D5 and the inner diameter D2 of the shaft thread is the same as the inner diameter D4 of the tip thread.

Alternatively, the inner diameter D2 of the shaft thread can be the same as the outer diameter D5 of the tip thread. This has the advantage that the screw can be screwed relatively easily into a pre-drilled pilot hole, because the tip encounters little friction.

The tip can be self-tapping. This has the advantage that a corresponding screw can also be used without a pilot hole.

The shaft can be made of titanium or stainless steel. These two materials are commonly used for bone implants. They show excellent mechanical stability and biocompatibility.

The shaft can be subjected to a surface finishing method. In particular if the shaft is made of stainless steel, it can be subjected to electropolishing. Electropolishing, also known as electrochemical polishing, anodic polishing, or electrolytic polishing, is an electrochemical process that removes material from a metallic workpiece, reducing the surface roughness by levelling micro-peaks and valleys, improving the surface finish. It is used to polish, passivate, and deburr metal parts. Electropolishing creates a clean, smooth surface that is easier to sterilize.

Alternatively, in particular if the shaft is made of titanium, it can be subjected to anodizing. Anodizing is an electrolytic passivation process used to increase the thickness of the natural oxide layer on the surface of metal parts. Anodizing increases resistance to corrosion and wear and also reduces the surface roughness.

Only part of the shaft's surface can be subjected to a finishing process reducing its roughness. In a preferred embodiment, only an outer surface of the shaft, which excludes at least an inner surface of the recess, is subjected to a finishing process reducing its roughness, in particular electropolishing and/or anodizing.

The inner surface of the recess, on the other hand, can be subjected to a finishing process increasing its roughness, such as sand blasting.

In a preferred embodiment, an outer surface of the shaft, which excludes at least an inner surface of the recess, is subjected to a finishing process reducing its roughness, such as electropolishing and/or anodizing, while the inner surface of the recess is subjected to a finishing process increasing its roughness, such as sand blasting. In such an embodiment, the outer surface of the shaft, excluding at least the inner surface of the recess, can have a roughness which is lower than the roughness of the inner surface of the recess. In particular, the outer surface of the shaft, excluding at least the inner surface of the recess, can have roughness value Rₐ of less than 3.2 μm. At the same time, the inner surface of the recess can have a Roughness value Rₐ of more than 3.2 μm.

In an alternative embodiment, the entire surface of the shaft can have the same roughness, for instance with a roughness value Rₐ between 0.8 to 3.2 μm.

The tip can be made of an absorbable polymer material. The absorbable polymer material can be selected from the group consisting of poly(DL-lactide), poly(L-lactide), poly(glycolide), poly(L-lactide-co-glycolide), poly(L-lactide-co-ε-caprolactone), poly(L-lactide-co-trimethylene carbonate), poly(L-lactide-co-PEG) triblock, poly(L-lactide-co-D,L-lactide), poly(L-lactide-co-D,L-lactide-co-PEG) triblock, poly(DL-lactide-co-glycolide), poly(ethyleneglycol)-co-polylactide, poly(caprolactone), poly(glycolide-co-ε-caprolactone), poly(glycolide-co-trimethylene carbonate), polydioxanone and mixtures thereof.

These polymer materials have the advantage that robust quality and safety data are available for them. They offer a wide range of degradation profiles and have tailorable mechanical properties. Furthermore, they are easy to process and stable after sterilization, have good mechanical properties and good biocompatibility.

The absorbable polymer material can have a degradation time, when implanted into a bone, of 3 years to 1 month, preferably 3 to 12 months.

Preferred polymer materials are selected from the group consisting of poly(D,L-lactide), poly(L-lactide-co-PEG) triblock, poly(L-lactide-co-D,L-lactide-co-PEG) triblock, poly(L-lactide-co-D,L-lactide-co-PEG) triblock and mixtures thereof.

The absorbable polymer material can additionally comprise at least one filler, such as hydroxylapatite or β-tricalcium phosphate. One advantage of these fillers is that they increase the radiodensity of the tip, which facilitates X-ray diagnostics. A further advantage is that they can increase the compressive strength of the tip. Moreover, they can decrease its degradation time.

Alternatively, the tip can also be made of a natural material, in particular a material selected from the group consisting of methylcellulose, carboxymethylcellulose, hyaluronic acid, chitosan, collagen, gelatin, fibrin, dextran and agarose.

As a further alternative, the tip can also be made of a metal, for instance magnesium.

A proximal end of the shaft can comprise a screw head. The screw head can be threaded or non-threaded. If the head is threaded, the head thread defines a third lead length and a third pitch. An advantage of having a threaded screw head is that the head may in this manner be locked in a desired angle within an opening or hole of a bone plate.

Preferably, the lead lengths of the shaft thread and the head are the same or substantially the same. This has the advantage that the screw can be locked with a bone plate with any damage caused to the bone tissue minimized or avoided.

The shaft thread can have an outer diameter D3 of 2.0 mm to 7.0 mm, preferably 2.5 mm to 5 mm, even more preferably 2.5 mm, 3.0 mm, 3.5 mm, 4.0 mm, 4.5 mm or 5.0 mm. The shaft can have a length L1 of 5 mm to 150 mm, preferably 10 mm to 100 mm. These are typical dimensions for orthopedic screws used in fixation of proximal humerus fractures.

The tip can have a length L2 of 2 mm to 20 mm, preferably 3 mm to 15 mm, even more preferably 4 to 12 mm. It has been found that the mentioned lengths result in good stability of proximal humerus fractures in the initial healing phase, while the risk of secondary screw penetration is substantially reduced.

In order to avoid any misunderstandings, if presently reference is made to the length L2 of the tip, this is without any additional length L3 of any protrusion of the tip, adapted for entering a recess of the shaft. In other words, the length L2 of the tip is typically the full length of the screw minus the length L1 of the shaft.

The protrusion of the shaft and/or tip can have a length L3 of 2 mm to 20 mm, preferably 3 mm to 15 mm, even more preferably 4 to 12 mm. At the same time, the recess of the shaft and/or tip can a depth L4 of 2 mm to 20 mm, preferably 3 mm to 15 mm, even more preferably 4 to 12 mm. With these dimensions, secure mounting of the tip on the shaft can be ensured.

In a preferred embodiment, the protrusion of the shaft and/or tip has a length L3 which is at least 50%, preferably, at least 60%, more preferably at least 70%, even more preferably at least 80%, even still more preferably at least 90%, of the depth L4 of the recess of the shaft and/or tip.

The recess can have a diameter D6 of 1.0 mm to 10 mm, depending on the inner diameter of the shaft thread. A larger diameter D6 of the recess can be preferred, as it allows making the tip and its protrusion with an essentially constant diameter over their entire length. This is particularly beneficial, if the tip is injected onto the shaft, since with such a geometry, improved flow properties of the molding material can be achieved. Nevertheless, D6 should not be too large, as a certain wall thickness of the recess is required.

The diameter D6 of the recess can be smaller than the inner diameter D2 of the threaded shaft, preferably by 0.8 mm, more preferably by 0.6 mm, even more preferably by 0.4 mm. This ensures sufficient wall thickness in all parts of the recess.

Preferably, the diameter D6 of the recess is smaller than the depth L4 of the recess.

The at least one undercut, in particular the circumferential groove, can have a depth U of 0.1 mm to 2.0 mm, preferably 0.2 mm to 1.5 mm, even more preferably 0.3 mm to 1.2 mm.

These depths ensure for secure attachment of the tip to the shaft, while they still allow different kinds of attachment, such as a snap fitting.

A further aspect of the present disclosure relates to a method for manufacturing an orthopedic screw, in particular an orthopedic screw as described herein above. The method comprises the steps of:
Providing a threaded shaft made of a non-absorbable material;
Providing a tip made of an absorbable material;
Assembling the shaft and the tip such that they are positively connected.

This method has the advantage that the shaft and the tip can be manufactured and sterilized separately. Furthermore, both components can be produced by conventional means, for instance the shaft by machining and the tip by injection molding. Assembly of the screw can be done either manually or automatically by conventional means. The whole manufacture process is therefore technically relatively undemanding.

As mentioned herein above, the shaft and the tip can be connected to each other through a snap fit connection and/or by ultrasonic welding.

Yet another aspect of the present disclosure relates to an alternative method for manufacturing an orthopedic screw, in particular an orthopedic screw as described herein above. The method comprises the steps of:
Providing a threaded shaft made of a non-absorbable material;
Providing an absorbable material;
Injecting the absorbable material upon the shaft to form a tip, such that the shaft and the tip are positively connected.

Injection molding the tip onto the shaft has the advantage that an additional manufacture step of assembling the screw is not required. The process is therefore particularly attractive for mass production of orthopedic screws in high numbers. Furthermore, injection molding the tip onto the shaft allows for deeper undercuts to secure the tip on the shaft in a positive connection, at least compared to a snap fit connection. A positive connection between the shaft and the tip also allows for a certain shrinkage of the tip material after injection molding.

When injection molding the tip onto the shaft, a shaft comprising at least one, preferably a plurality of, ventilation holes and/or injection points (as described herein above) is preferred. The use of ventilation holes and/or several injection points is important, because air inclusions can occur in the injection molded material. Such air inclusions are normally relatively small and pose no problem per se to the mechanical stability of the tip. However, air inclusions can lead to local overheating of the material, which can result in reduced bioabsorption times (i.e. degradation times).

The above method, involving injection molding, can further comprise the step of positioning the shaft in an injection molding machine. A tight connection can be created between the shaft of the screw and an injection mold, preferably at a sealing surface, which is in particular a circumferential area at a distal end of the shaft, having a cylindrical or conical shape, preferably cylindrical. Also, sealing surfaces surrounding ventilation holes and/or injection points can be beneficial (as described herein above). Furthermore, the injecting can be effected in a longitudinal and/or transversal and/or oblique direction, relative to the shaft. If a transversal and/or oblique direction is chosen, the injection can occur through the injection points.

The injection mold used can have one or a plurality of cavities. The cavities can have various lengths, in order to form tips of various lengths. The injection mold can consist of two mold halves. The angular position of the shaft in the mold can for instance be controlled over the screw driving profile (e.g. Torx or hexagon) at the screw's head. Alternatively, the shaft can comprise one or more flat surfaces at its side, for instance interrupting the threads, or it can comprise a transversal recess or hole for this purpose.

A further aspect of the present disclosure relates to an orthopedic screw obtainable by a method as described herein above.

Yet another aspect of the present disclosure relates to a bone plate system comprising a bone plate and at least one orthopedic screw, as described herein above.

The bone plate can comprise at least one, preferably a plurality of, holes. The holes may be threaded or non-threaded (before they receive the bone screws). If the holes are non-threaded, then the screws or at least their head portions may be made of harder material than the material of the walls of the holes. In this manner, the screws may be designed to form a female thread in the hole walls. Furthermore, the material of the bone plate may be selected so that the threads can be formed irreversibly. The advantage of forming the threads is that the screws can in this manner be locked in the bone plate in any desired angular orientation.

It is to be understood that both the foregoing general description and the following detailed description present embodiments are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF THE DRAWINGS

The herein described disclosure will be more fully understood from the detailed description given herein below and the accompanying drawings which should not be considered limiting to the invention described in the appended claims. The drawings are showing:

FIG. 1a shows a perspective view of an orthopedic screw according to a first embodiment of the present disclosure;

FIG. 1b shows a side view of the orthopedic screw according to FIG. 1a;

FIG. 2a shows a cross-sectional view of an orthopedic screw according to FIGS. 1a and 1b, along a longitudinal axis A1 of the screw;

FIG. 2b shows a close-up of FIG. 2a;

FIG. 2c shows the close-up of FIG. 2b, with the shaft and the tip separated;

FIG. 2d shows a further close-up of FIG. 2a;

FIG. 3a shows a perspective view of an orthopedic screw according to a second embodiment of the present disclosure;

FIG. 3b shows a close-up cross-sectional view of an orthopedic screw according to FIG. 3a;

FIG. 3c shows a cross-sectional view of an orthopedic screw according to a variation of the embodiment of FIGS. 3a and 3b;

FIG. 4a shows a close-up cross-sectional view of a third embodiment of the present disclosure;

FIG. 4b shows a cross-sectional view of an orthopedic screw according to a variation of the embodiment of FIG. 4a;

FIG. 5 shows a close-up cross-sectional view of a fourth embodiment of the present disclosure;

FIG. 6 shows a close-up cross-sectional view of a fifth embodiment of the present disclosure;

FIG. 7a shows a cross-sectional view of a sixth embodiment of the present disclosure;

FIG. 7b shows a close-up of FIG. 7a;

FIG. 8a shows a cross-sectional view of a seventh embodiment of the present disclosure;

FIG. 8b shows a close-up of FIG. 8a;

FIG. 9 shows a close-up cross-sectional view of an eighth embodiment of the present disclosure;

FIG. 10a shows a perspective view of an orthopedic screw according to a ninth embodiment of the present disclosure;

FIG. 10b shows a cross-sectional close-up of an orthopedic screw according to FIG. 10a;

FIG. 11 shows a cross-sectional close-up of an orthopedic screw according to a tenth embodiment of the present disclosure;

FIG. 12a shows a perspective view of an orthopedic screw according to an eleventh embodiment of the present disclosure;

FIG. 12b shows a cross-sectional close-up of an orthopedic screw according to FIG. 12a;

FIG. 13 shows a cross-sectional close-up of an orthopedic screw according to a twelfth embodiment of the present disclosure;

FIG. 14 shows a bone plate system comprising a set of orthopedic screws implanted in a target bone and before the tip of the screws have degraded;

FIG. 15 shows a perspective exploded sectional view of an orthopedic screw according to a thirteenth embodiment of the present disclosure with partial enlargement;

FIG. 16 shows a perspective view of the shaft of an orthopedic screw according to FIG. 15 with partial enlargement;

FIG. 17 shows a perspective exploded sectional view of an orthopedic screw according to a fourteenth embodiment of the present disclosure with partial enlargement;

FIG. 18 shows a perspective view of the shaft of an orthopedic screw according to FIG. 17 with partial enlargement;

FIG. 19 shows a perspective exploded sectional view of an orthopedic screw according to a fifteenth embodiment of the present disclosure with partial enlargement;

FIG. 20 shows a perspective exploded sectional view of an orthopedic screw according to a sixteenth embodiment of the present disclosure with partial enlargement;

FIG. 21 shows a perspective exploded sectional view of an orthopedic screw according to a seventeenth embodiment of the present disclosure;

FIG. 22 shows a perspective exploded sectional view of an orthopedic screw according to an eighteenth embodiment of the present disclosure;

FIG. 23 shows a side view of the shaft of an orthopedic screw according to a nineteenth embodiment of the present disclosure with partial enlargement;

FIG. 24 shows a side view of the shaft of an orthopedic screw according to a twentieth embodiment of the present disclosure with partial enlargement;

FIG. 25 shows a side view of the shaft of an orthopedic screw according to a twenty-first embodiment of the present disclosure with partial enlargement;

FIG. 26 shows a perspective view of an orthopedic screw according to a twenty-second embodiment of the present disclosure;

FIG. 27 shows a perspective exploded sectional view of an orthopedic screw according to FIG. 26.

### DETAILED DESCRIPTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

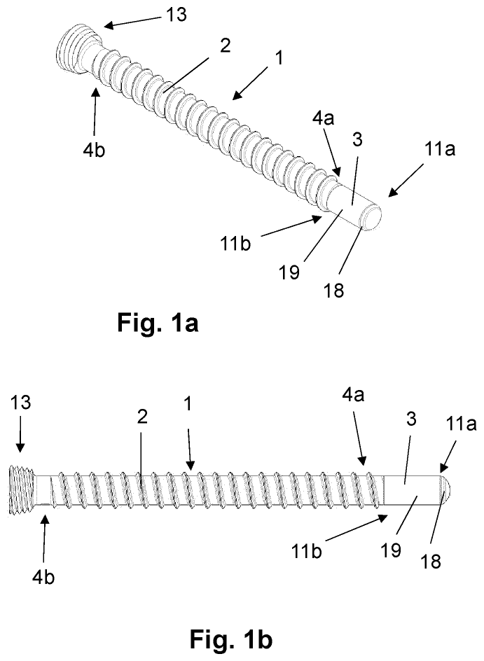

FIGS. 1a and 1b show an orthopedic screw 1 according to a first embodiment of the present disclosure. The orthopedic screw 1 comprises a threaded shaft 2 made of a non-absorbable material and a tip 3 at a distal end 4a of the shaft 2 made of an absorbable material. A proximal end 4b of the shaft 2 comprises a threaded screw head 13. The distal end 11a of the tip comprises a rounded or curved surface 18 which extends towards the proximal end 11b of the tip to merge with a cylindrical section 19.

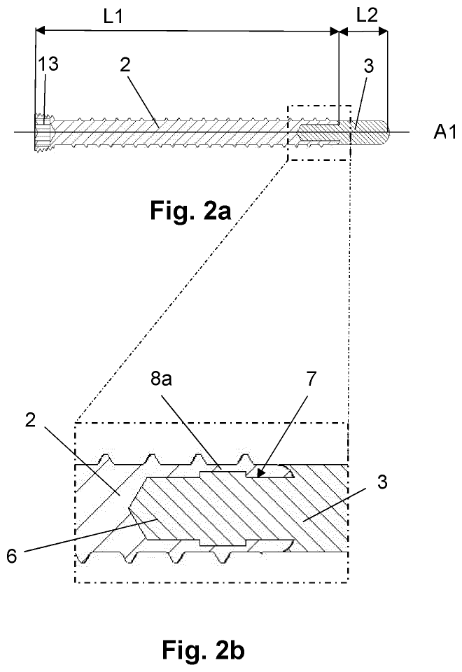

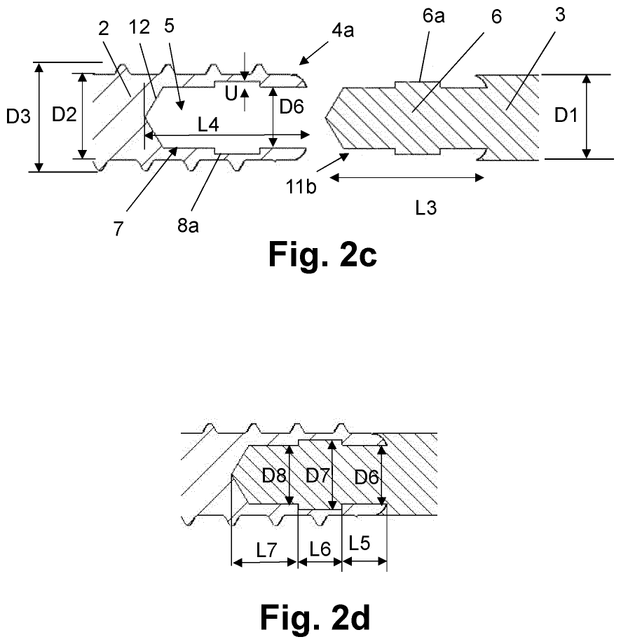

Referring to FIG. 2a, the threaded shaft 2 has a length L1 and the tip 3 a length L2. The overall length of the screw is the sum of L1 and L2. Though not visible from this general view, the shaft 2 and the tip 3 are positively connected as described in FIGS. 2b to 2d below.

From the close-ups of FIGS. 2b to 2d, further details of the connection between the shaft 2 and the tip 3 can be discerned. Referring to FIGS. 2b and 2c, there is a protrusion 6 at the proximal end 11b of the tip 3 and a corresponding recess 5 at the distal end 4a of the shaft 2, into which the protrusion can engage to establish a positive connection. The protrusion has a length L3, which is the same as a depth L4 of the recess 5. The cross sections of the recess 5 and the protrusion 6, taken orthogonally to the longitudinal axis A1 of the screw 1, are circular. The shaft thread has an inner diameter D2 and an outer diameter D3. In the shown embodiment, the diameter D1 of the tip equals the inner diameter D2 of the shaft thread. The diameter D6 of the recess 5 of the shaft 2 (at its mouth) is smaller than the inner diameter D2 of the shaft thread, typically by at least 0.2 mm, in order provide sufficient structural stability. In this embodiment, the diameter D6 at the mouth of the recess 5 is also the diameter throughout the recess 5 along the longitudinal axis A1. In other words, the recess 5 and the protrusion 6 extend along the longitudinal axis A1 of the screw 1. The distal end 4a of the threaded shaft 2 is rounded. This means that even after the tip 3 has degraded, the screw 1 still has rounded edges or edge surfaces, at least at the distal end 4a of its shaft 2. Furthermore, as a transitional surface between tip and the shaft is curved and therefore longer, bioabsorption at the connection between the tip and the shaft can be slowed.

A side wall 7 of the recess 5 comprises an undercut 8a. The undercut 8a lies in depth U from the diameter D6 of the recess 5 of the shaft 2 and extends circumferentially along the entire circumference of the side wall 7. The undercut 8a is shaped and sized to engage with an engagement element on the protrusion 6 of the tip 3. In the present example, the engagement element has the shape of an increased-diameter portion 6a of the protrusion 6. The end 12 of the recess has a conical shape.

As shown in FIG. 2d, the undercut 8a lies in a depth L5 from the mouth of the recess 5. The undercut 8a has a width L6. Furthermore, the recess has a depth L7 proximal to undercut 8a. In the present embodiment, L5 and L6 are the same, L7 is slightly larger. The diameter D7 of the recess 5 at the undercut 8a is slightly larger than its diameter D6 at its mouth, but smaller than the inner diameter of the threaded shaft D2. The diameter D8 of the recess 5 proximal to the undercut 8a is equal to the diameter at the mouth of the recess D6.

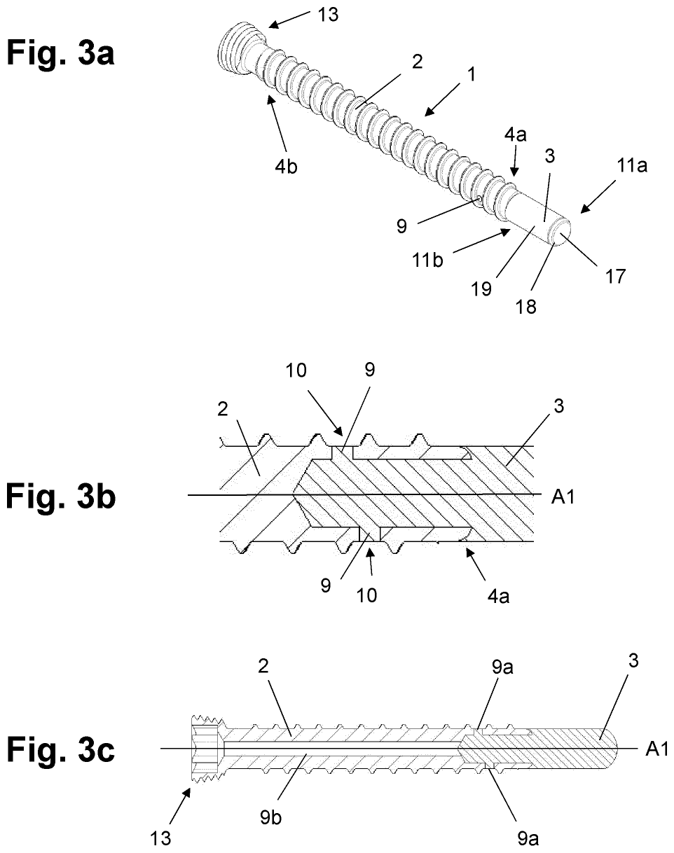

In a second embodiment shown in FIGS. 3a and 3b, ventilation holes and/or injection points 9 are inserted into the grooves 10 of the threaded shaft 2. These ventilation holes and/or injection points 9 are oriented perpendicular to longitudinal axis A1. As apparent from FIG. 3b, the ventilation holes and/or injection points 9 form part in the positive connection. The distal end 11a of the tip comprises a flat surface 17. This surface 17 is surrounded by a rounded or curved surface 18, which extends from the flat surface 17 towards the proximal end 11b of the tip to merge with a cylindrical section 19.

FIG. 3c shows a variation of the second embodiment discussed in the preceding paragraph. In addition to transversal ventilation holes and/or injection points 9a, in this embodiment, the shaft 2 of the screw additionally comprises a longitudinal ventilation hole 9b, which extends from the proximal end of the recess 5 along the longitudinal axis A1 to the screw head 13. The screw 1 is thus partially cannulated.

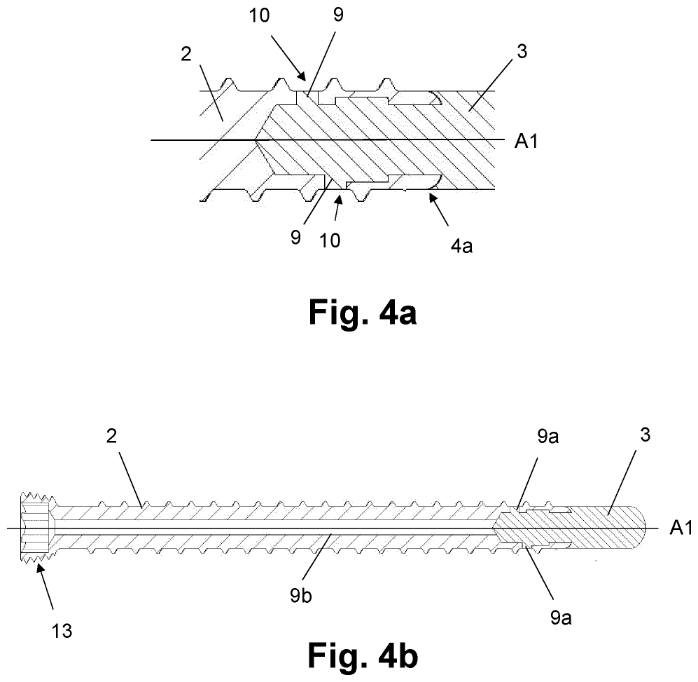

FIG. 4a shows a third embodiment, where undercuts are present in addition to the ventilation holes and/or injection points 9. FIG. 4b shows a variation of this third embodiment, which comprises a longitudinal ventilation hole 9b, analogous to the variation shown in FIG. 3c.

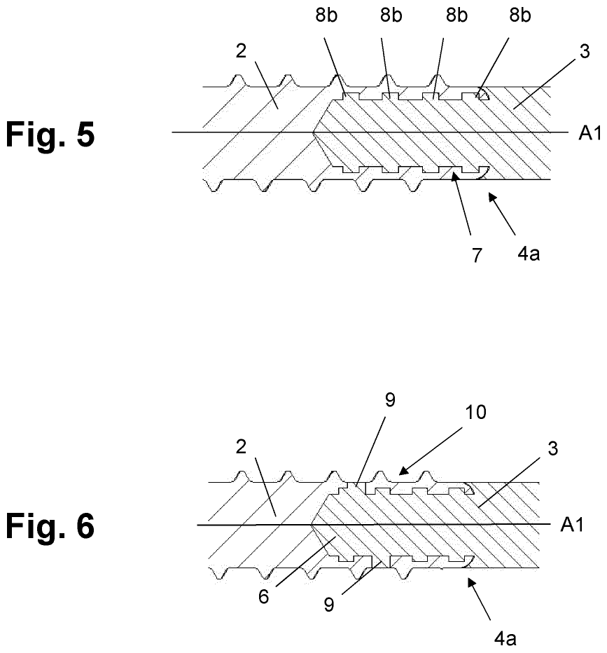

Turning to FIG. 5, a fourth embodiment of the present disclosure is shown with several grooves 8b instead of one single undercut 8a at the inner wall 7 of the recess 5. These are distinct circumferential grooves, but also a threading is possible.

A fifth embodiment is shown in FIG. 6, which includes ventilation holes and/or injection points 9 in combination with a plurality of grooves 8b.

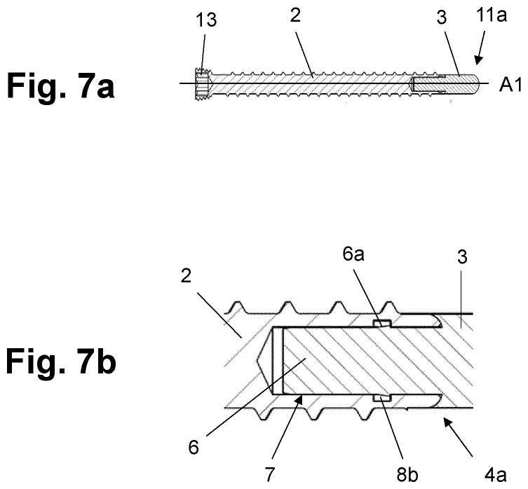

FIGS. 7a and 7b show a sixth embodiment of the present disclosure, where the tip 3 is connected to the shaft 2 through a snap fitting. In this example, the side wall 7 of the recess 5 comprises a circumferential groove 8b shaped and sized complementary to engage with the increased-diameter portion 6a of the protrusion 6. In the present cross-sectional view, the increased-diameter portion 6a has the form of a wedge with an increased diameter towards the distal end of the tip 2. When coupling the tip 3 to the shaft 2, the side wall 7 of the shaft 2 may deform or flex outwardly or outwards when it comes in contact with the protrusion 6 of the tip 3, and resile inwardly or inwards when the groove 8b becomes engaged with the increased-diameter portion 6a. In addition, or alternatively, the increased-diameter portion 6a may deform inwardly when the side wall 7 comes in contact with the increased-diameter portion 6a, and resile outwardly when the circumferential groove 8b is engaged with the increased-diameter portion 6a. Thus, the side wall 7 and or/the increased-diameter portion 6a may be configured as an elastically deformable element.

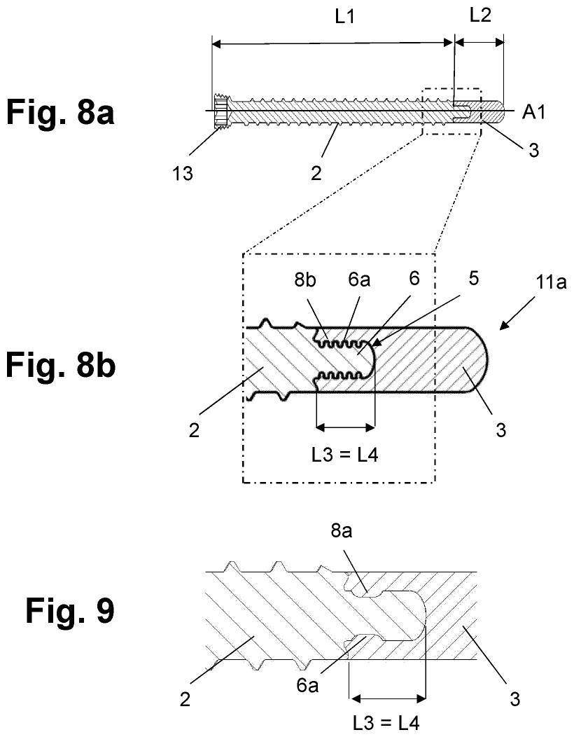

Turning to FIGS. 8a, 8b and 9, a seventh and eighth embodiment with a recess 5 in the tip 3 and a protrusion 6 in the shaft 2 is shown. The length L3 of the protrusion 6 equals the length L4 of the recess 5. The sidewall of the recess comprises plurality of circumferential grooves 8b or undercuts 8a. The protrusion 6 of the shaft 2 comprises a plurality of corresponding engagement elements 6a. FIG. 8b shows the eighth embodiment with circumferential grooves 8b, FIG. 9 shows the ninth embodiment with undercuts 8a.

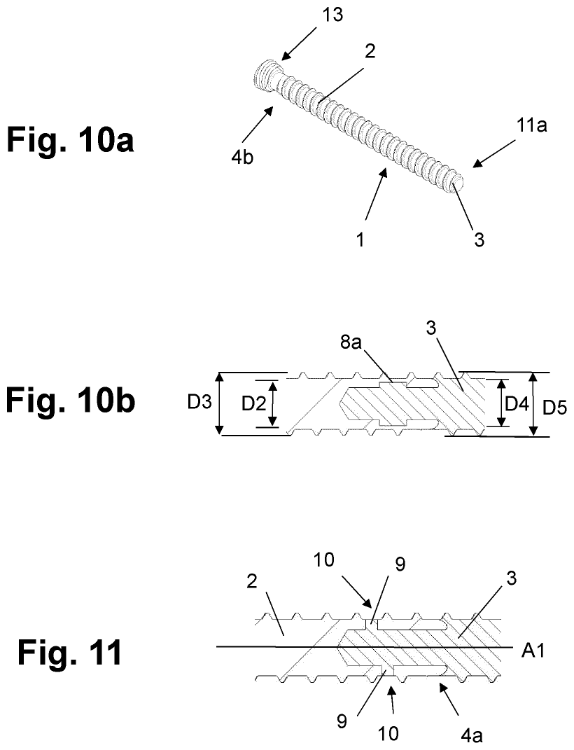

FIGS. 10a, 10b and 11 show a ninth and tenth embodiment of an orthopedic screw 1 according to the present disclosure. Other than the previously shown embodiments, the tip 3 is threaded. The inner diameter D4 of the tip thread corresponds to the inner diameter D2 of the shaft thread. The outer diameter D5 of the tip thread corresponds to the outer diameter D3 of the shaft thread. The lead length of the tip is the same as the lead length of the shaft. The thread of the tip is aligned with the thread of the shaft. It is to be noted that as the screw 1 according to the present embodiment is not self-tapping, a pilot hole is needed to implant this screw 1. In order to ensure that shaft 2 and tip 3 being positively connected, an undercut 8a, circumferential grooves 8b or ventilation holes and/or injection points 9 might be introduced individually or combined with each other in an embodiment. An undercut 8a is shown in in FIG. 10b. As before, also a plurality of circumferential grooves are conceivable. Furthermore, FIG. 11 shows ventilation holes and/or injection points 9.

In the present embodiment, rotation between the shaft 2 and the tip 3 around the longitudinal axis A1 of the screw needs to be locked, for instance with a polygonal recess/protrusion, in order to drive rotation of the tip preserve alignment of the threads.

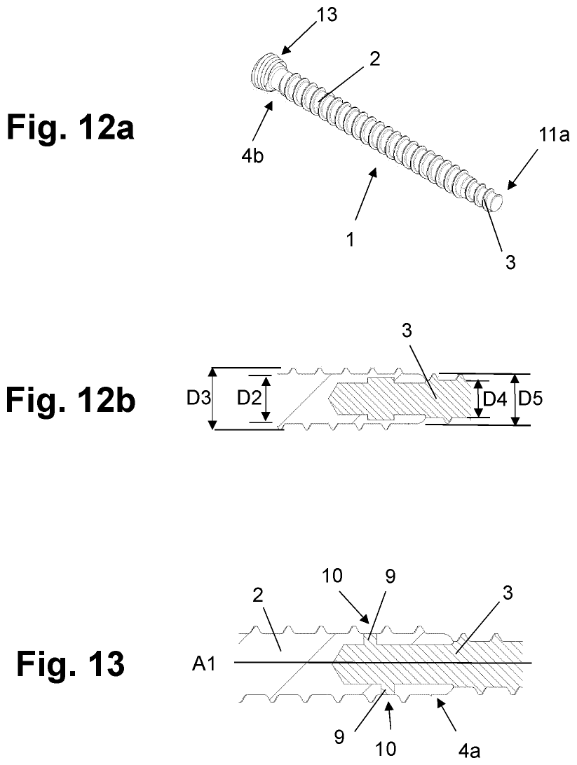

FIGS. 12a, 12b and 13 show an eleventh and twelfth embodiment of the orthopedic screw 1. Similar to FIGS. 10 and 11, the tip 3 is threaded, but in this embodiment, the outer diameter D5 of the tip thread equals the inner diameter D2 of the shaft thread. Again, rotation between the shaft 2 and the tip 3 around the longitudinal axis A1 of the screw needs to be locked, in order to drive rotation of the tip. Again, in order to ensure that shaft 2 and tip 3 being positively connected, an undercut 8a, circumferential grooves 8b or ventilation holes and/or injection points 9 might be introduced individually or combined with each other in an embodiment.

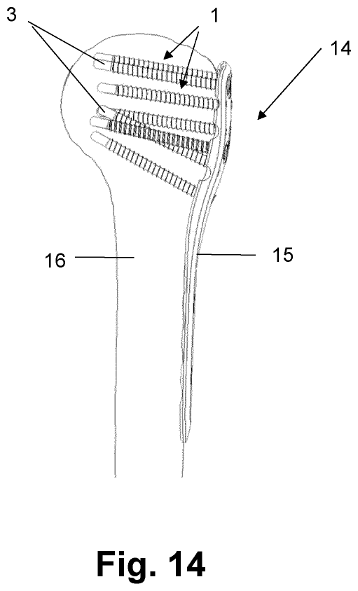

FIG. 14 shows an exemplary arrangement of orthopedic screws 1 in a bone plate assembly 14 in a target bone 16, for instance a humerus bone in the shoulder joint. The orthopedic screws 1 fasten a bone plate 15 onto the bone, forming the bone plate assembly 14. The tips 3 of the orthopedic screws 1 penetrate as far as possible into the bone tissue adjacent to the humeral head for improved fixation of the bone plate assembly 14. In the figure, the tips 3 of the screws 1 have not yet degraded. The requirement of improved fixation is even more important in case the patient is osteoporotic. The fact that the tip 3 of the screws is absorbable will prevent secondary screw penetration in case of bone sintering.

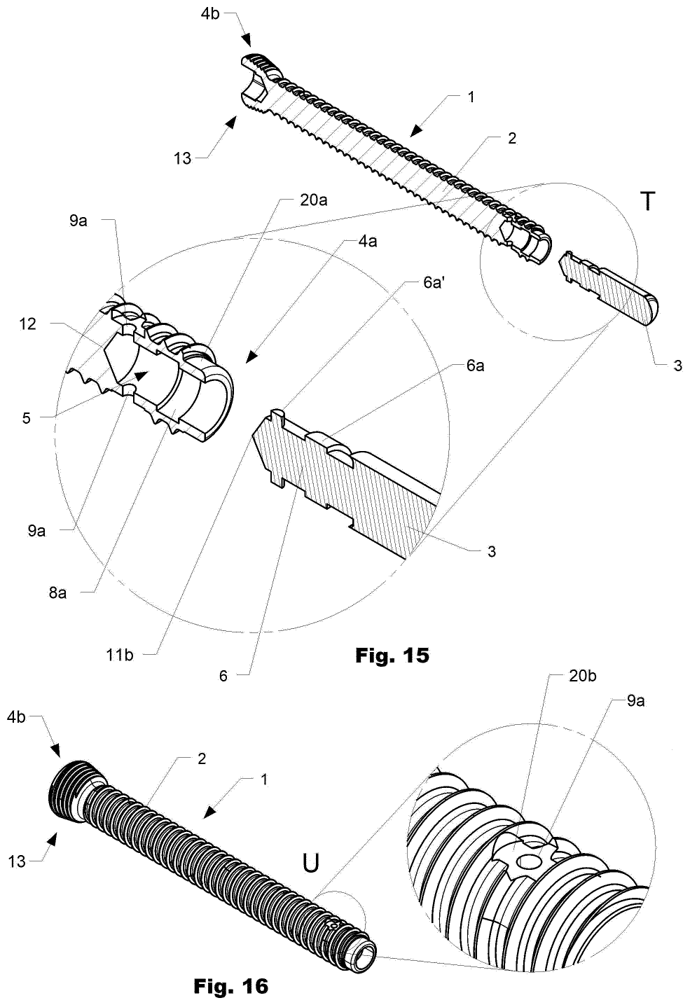

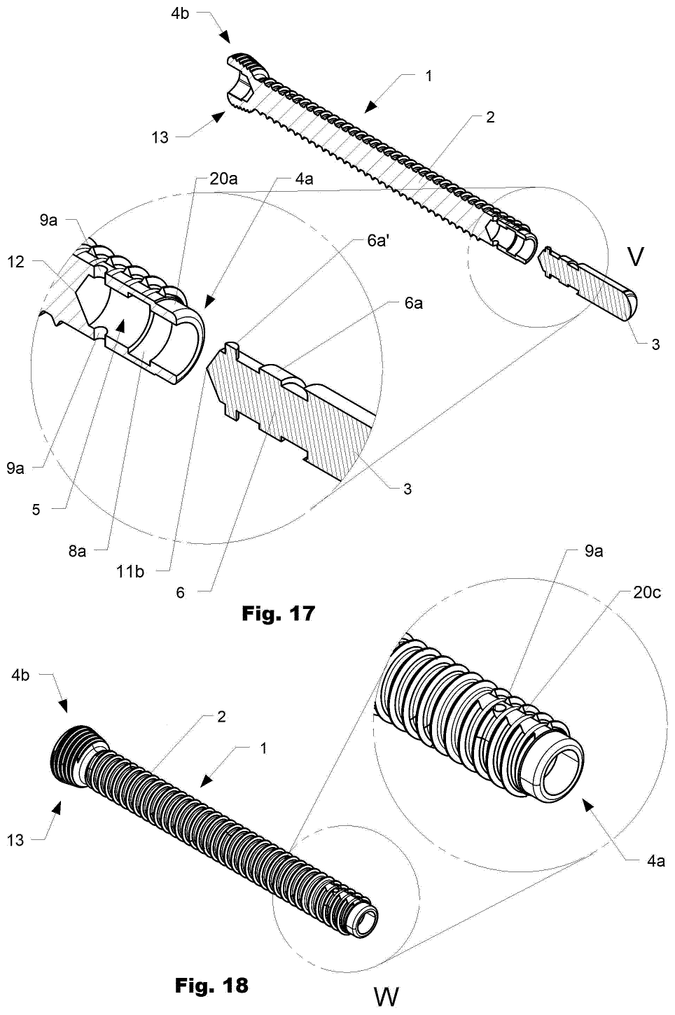

FIGS. 15 to 18 show a thirteenth and fourteenth embodiment of an orthopedic screw 1 according to the present disclosure, which are similar to the embodiment according to FIGS. 4a and 4b. In this embodiment, the tip 3 is formed on the shaft 2 by injection molding. This means that although the shaft 2 and the tip 3 are presently shown in exploded view, they normally do not exist as separate parts. In order to inject the absorbable material of the tip 3 into the recess 5, which is usually effected from the distal end 4a of the shaft 2, the shaft 2 comprises two transversal ventilation holes 9a, which are coaxial with each other (see enlarged parts T and V). The ventilation holes 9a are filled with absorbable material, which there forms engagement elements 6a′. These engagement elements 6a′ not only establish a positive connection between the tip 3 and the shaft 3, they also lead to a rotational locking.

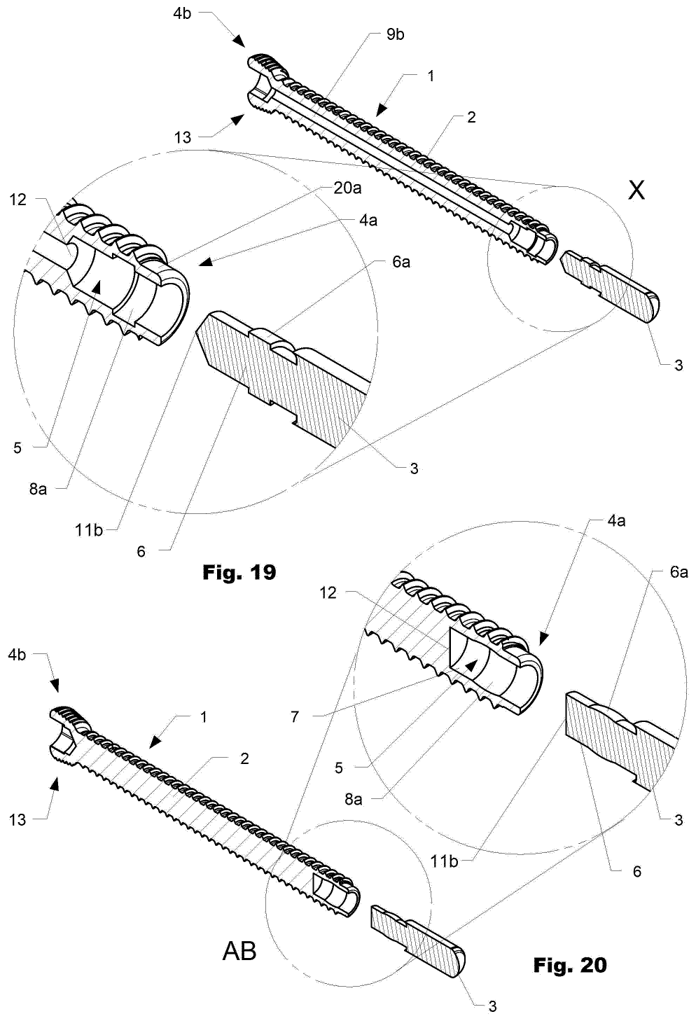

In order to establish a tight connection between the shaft 2 and an injection mold (not shown), there is a cylindrical sealing surface 20a at the distal end 4a of the shaft 2. Also the ventilation holes 9a are surrounded by sealing surfaces 20b or 20c, respectively. In the enlarged part U of FIG. 16, the sealing surface 20b is planar and cross-shaped around the respective ventilation hole 9a. In the enlarged part W of FIG. 18, the sealing surface 20c is formed by the base of a u-shaped longitudinal groove extending substantially parallel to the center axis A1 of the screw. FIG. 19 shows an orthopedic screw 1 according to a fifteenth embodiment of the present disclosure, which comprises a longitudinal ventilation hole 9b. It is therefore similar to the variation of the second embodiment according to FIG. 3c. In this embodiment, the shaft 2 comprises solely a longitudinal ventilation hole 9b. The screw 1 is thus partially cannulated.

FIG. 20 shows an orthopedic screw 1 according to a sixteenth embodiment of the present disclosure. In this embodiment, the tip 3 is mounted on the shaft 2 through a snap fitting, similar to FIGS. 7a and 7b. The increased-diameter portion 6a has a rounded form and the side wall 7 of the recess 5 comprises a respective undercut. When coupling the tip 3 to the shaft 2, the side wall 7 of the shaft 2 may deform or flex outwardly or outwards when it comes in contact with the protrusion 6 of the tip 3, and resile inwardly or inwards when the undercut 8a becomes engaged with the increased-diameter portion 6a. In addition, or alternatively, the increased-diameter portion 6a may deform inwardly when the side wall 7 comes in contact with the increased-diameter portion 6a, and resile outwardly when the undercut 8a is engaged with the increased-diameter portion 6a. Thus, the side wall 7 and or/the increased-diameter portion 6a may be configured as an elastically deformable element.

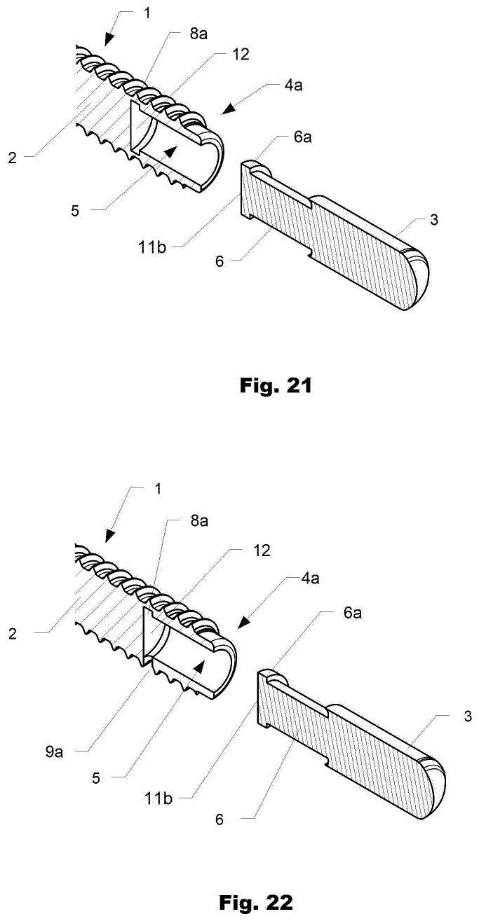

FIGS. 21 and 22 show an orthopedic screw 1 according to a seventeenth and eighteenth embodiment of the present disclosure. In these embodiments, the tip 3 is mounted to the shaft 2 by ultrasonic welding. To this end, a protrusion 6 of the tip 3 is inserted into a recess 5 of the shaft 2 and ultrasonic energy is introduced into the tip, in order to melt it at its proximal end 11b. An undercut 8a at the end 12 of the recess 5 is thus filled with absorbable material, and the increased-diameter portion 6a is formed. The embodiments according to FIGS. 21 and 22 are essentially identical. However, the embodiments according to FIG. 22 additionally comprises a ventilation hole 9a. In both figures, the tip 3 is shown as it looks after ultrasonic welding, i.e. with increased-diameter portion 6a, which does not exist beforehand.

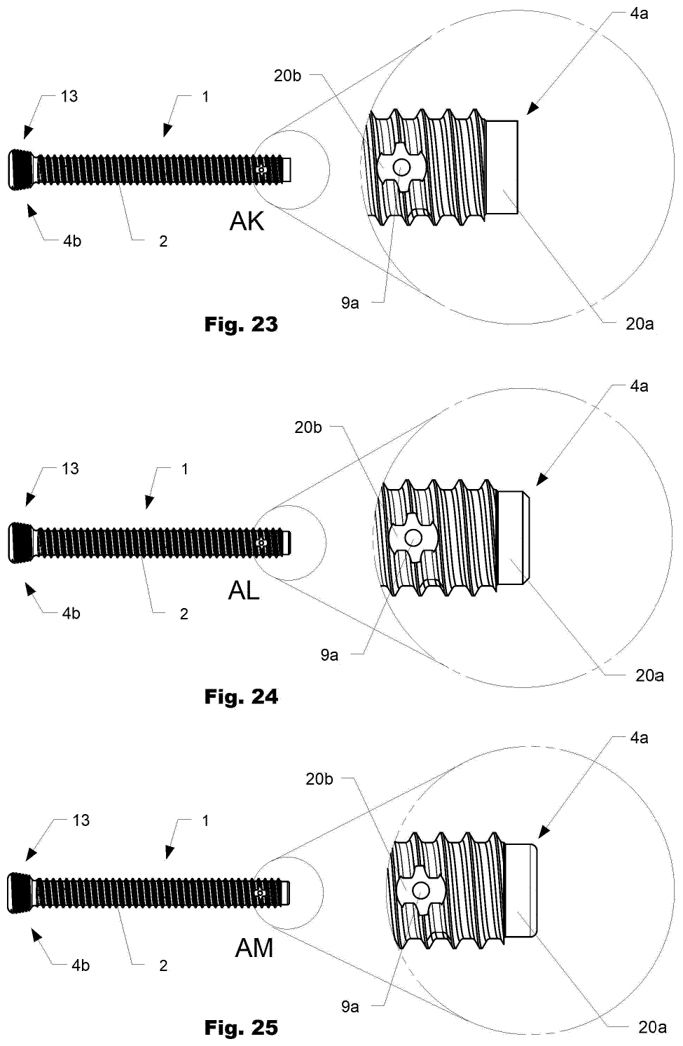

FIGS. 23 to 25 show different variations of the shaft 2 of an orthopedic screw 1 according to the present disclosure. As apparent, the distal end 4a of the shaft 2 can either be edged (FIG. 23), chamfered (FIG. 24) or rounded (FIG. 25).

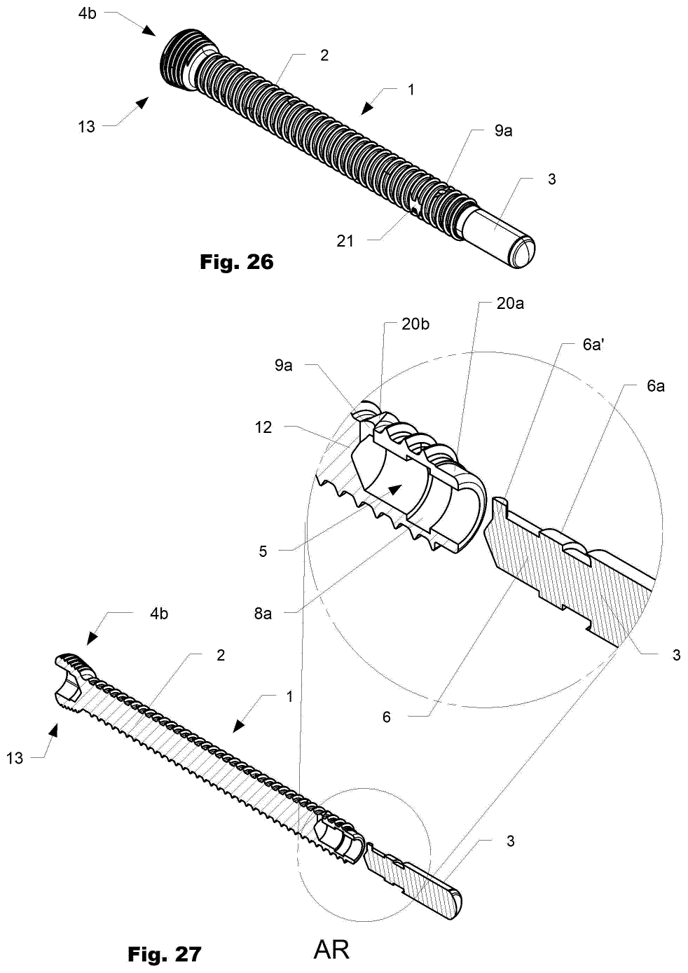

FIGS. 26 and 27 show an orthopedic screw 1 according to a twenty-second embodiment of the present disclosure. In this embodiment, the shaft 2 comprises a flat surface 21 on its side, which is interrupting the threading. This surface 21 has the purpose to angularly align the shaft 2, when it is put into an injection mold for forming the tip 3. From the enlarged part AR of FIG. 27 further details of the design of the screw 1 are apparent. It can for instance be seen that the sealing surface 20b around the ventilation hole 9a is implemented as a flat oval strip at the screw's thread.

The words used in this specification are words of description rather than limitation, and it is understood that various changes may be made without departing from the spirit and scope of the disclosure.

LIST OF DESIGNATIONS

| | |
|---|---|
| 1 | Orthopedic screw |
| 2 | Shaft |
| 3 | Tip |
| 4a | Distal end of the shaft |
| 4b | Proximal end of the shaft |
| 5 | Recess |
| 6 | Protrusion |
| 6a | Engagement element/increased-diameter portion |
| 7 | Side wall of recess |
| 8a | Undercut |
| 8b | Groove |
| 9 | Ventilation hole and/or injection point |
| 9a | Transversal ventilation hole and/or injection point |
| 9b | Longitudinal ventilation hole |
| 10 | Shaft thread grooves |
| 11a | Distal end of the tip |
| 11b | Proximal end of the tip |
| 12 | End recess |
| 13 | Screw head |
| 14 | Bone plate assembly or system |
| 15 | Bone plate |
| 16 | Target bone |
| 17 | Flat surface |
| 18 | Rounded surface |
| 19 | Cylindrical surface |
| 20a | Sealing surface (cylindrical) |
| 20b | Sealing surface (flat) |
| 20c | Sealing surface (base of u-shaped groove) |
| 21 | Flat surface on side of shaft |
| A1 | Longitudinal center axis |
| D1 | Diameter of the tip |
| D2 | Inner diameter of the shaft thread |
| D3 | Outer diameter of the shaft thread |
| D4 | Inner diameter of the tip thread |
| D5 | Outer diameter of the tip thread |
| D6 | Diameter recess |
| D7 | Diameter recess at undercut |
| D8 | Diameter recess proximal to undercut |
| U | Depth of undercut |
| L1 | Length shaft |
| L2 | Length tip |
| L3 | Length protrusion |
| L4 | Depth recess |
| L5 | Recess depth to undercut |
| L6 | Undercut width |
| L7 | Recess depth proximal to undercut |

1. An orthopedic screw, comprising
a threaded shaft made of a non-absorbable material and
a tip made of an absorbable material,
wherein the shaft and the tip are positively connected.

2. The orthopedic screw according to claim 1, wherein the shaft and the tip are additionally connected by a forced closure.

3. The orthopedic screw according to claim 1, wherein a distal end of the shaft comprises a recess into which a protrusion at a proximal end of the tip protrudes.

4. The orthopedic screw according to claim 3, wherein the recess extends along a longitudinal center axis of the shaft.

5. The orthopedic screw according to claim 3, wherein the recess is cylindrical, prismatic or conical in shape.

6. The orthopedic screw according to claim 3, wherein the recess comprises at least one undercut.

7. The orthopedic screw according to claim 6, wherein the recess is cylindrical and comprises at least one undercut in the form of circumferential grooves.

8. The orthopedic screw according to claim 7, wherein the number of circumferential grooves is 1, 2, 3, 4, 5, 6, 7, 8, 9 or 10.

9. The orthopedic screw according to claim 3, wherein the recess of the shaft comprises at least one selected from the group consisting of ventilation holes and injection points.

10. The orthopedic screw according to claim 9, wherein the at least one ventilation hole or injection point is oriented in a transversal or longitudinal direction, relative to the shaft.

11. The orthopedic screw according to claim 10, wherein the at least one ventilation hole or injection point is oriented in a transversal direction relative to the shaft and an area around the ventilation hole or injection point is devoid of screw threads, in order to form a sealing surface.

12. The orthopedic screw according to claim 1, wherein the shaft comprises a sealing surface in form of a circumferential area at a distal end of the shaft.

13. The orthopedic screw according to claim 1, wherein the tip is non-threaded.

14. The orthopedic screw according to claim 1, wherein the diameter of the tip is equal to or smaller than the inner diameter of the threaded shaft.

15. The orthopedic screw according to claim 1, wherein the distal end of the tip is rounded.

16. The orthopedic screw according to claim 1, wherein the shaft is made of titanium or stainless steel.

17. The orthopedic screw according to claim 1, wherein an outer surface of the shaft, excluding at least an inner surface of the recess, has a roughness which is lower than the roughness of the inner surface of the recess.

18. The orthopedic screw according to claim 1, wherein the tip is made of an absorbable polymer material.

19. The orthopedic screw according to claim 18, wherein absorbable polymer material is selected from the group consisting of poly(DL-lactide), poly(L-lactide), poly(glycolide), poly(L-lactide-co-glycolide), poly(L-lactide-co-ε-caprolactone), poly(L-lactide-co-trimethylene carbonate), poly(L-lactide-co-PEG) triblock, poly(L-lactide-co-D,L-lactide), poly(L-lactide-co-D,L-lactide-co-PEG) triblock, poly(DL-lactide-co-glycolide), poly(ethyleneglycol)-co-polylactide, poly(caprolactone), poly(glycolide-co-ε-caprolactone), poly(glycolide-co-trimethylene carbonate), polydioxanone and mixtures thereof.

20. A method for manufacturing an orthopedic screw, the method comprising:
Providing a threaded shaft made of a non-absorbable material;
Providing a tip made of an absorbable material; and
Assembling the shaft and the tip such that they are positively connected.

21. A method for manufacturing an orthopedic screw, the method comprising:
Providing a threaded shaft made of a non-absorbable material;
Providing an absorbable material; and
Injecting the absorbable material upon the shaft to form a tip, such that the shaft and the tip are positively connected.

22. (canceled)

23. (canceled)
